(19) United States
(12) Patent Application Publication
(10) Pub. No.: US 2007/0089740 A1
(43) Pub. Date: Apr. 26, 2007

(54) Pursed lip breathing device

(75) Inventors: Beth A. Baumert, Columbia, CT (US); Janice G. Proll, Andover, CT (US)

Correspondence Address: MCCORMICK, PAULDING & HUBER LLP, CITY PLACE II, 185 ASYLUM STREET, HARTFORD, CT, 06103, US

(73) Assignee: CHI, LLC, Columbia, CT (US)

(21) Appl. No.: 10/554,573

(22) PCT Filed: Apr. 28, 2004
(86) PCT No.: PCT/US04/12988

§ 371 Date: Oct. 27, 2005

Related U.S. Application Data
(60) Provisional application No. 60/465,988, filed on Apr. 28, 2003.

(51) Int. Cl. A61M 15/00

(52) U.S. Cl. 128/203.12; 128/200.24

(57) ABSTRACT

A pursed lip breathing device includes a housing defining an air channel through which a user breathes. A mouthpiece is attached to the housing and includes an aperture generally aligned with the air channel so that when the user breathes through the aperture, an airflow is generated in the air channel. The mouthpiece has outer dimensions designed so that the user's lips are pursed during breathing. During breathing, the airflow in the air channel is restricted to include pressure on the user's pulmonary system. Information or feedback regarding the user's breathing may also be provided so that the user is motivated and encouraged during breathing exercises and may monitor the therapeutic effect of his or her breathing.

### CROSS REFERENCE TO RELATED APPLICATION

This application is entitled to the benefit of and incorporates by reference essential subject matter disclosed in International Patent Application No. PCT/US2004/012988 filed on Apr. 28, 2004 and U.S. Provisional Application No. 60/465,988 filed Apr. 28, 2003.

### BACKGROUND OF THE INVENTION

The present invention relates to breathing devices, and, in particular, breathing devices used for respiratory therapy. More specifically, the present invention relates to a pursed lip Positive Expiratory Pressure (PEP) breathing device including a mouthpiece designed to cause the user to purse his or her lips during breathing exercises in order to maximize back pressure in the user's bronchial tubes and lungs.

Breathing exercises, with or without a breathing device, are indicated for patients with pulmonary conditions such as asthma, cystic fibrosis (CF), obstructive sleep apnea (OSA), COPD and/or other pulmonary disorders. Breathing exercises can also increase athletic ability and fitness, and decrease stress and tension, even in healthy individuals. For example, yoga breathing techniques may involve a series of relaxed, rhythmic, pressurized exhales through pursed lips. However, breathing exercises require significant self-discipline. Moreover, anyone with chronic breathing problems may experience difficulty performing these breathing exercises in much the same way that an arthritic would experience difficulty undergoing a jogging routine.

A “PEP” or “Positive Expiratory Pressure” breathing device makes breathing exercise and therapy easier to perform by supplying resistance for the breathing exercises, much in the same manner as fitness equipment in the gym provides isometric resistance for physical exercise and therapy. That is, when one exhales through a PEP breathing device, the airflow is restricted. This causes back-pressure—or “positive expiratory pressure”—in the bronchial airways and lungs that physically opens air passages and increases lung capacity by pressing or splinting the walls of the airways open in the same way that air pressure expands a balloon. As the pulmonary airways become splinted open, the resulting airflow promotes mobilization of mucus obstructions in the bronchial tubes. Coughing will often ensue and result in expelling obstructions after breathing exercises utilizing a PEP breathing device. Additionally, repetitive exercising with a PEP breathing device strengthens the pulmonary musculature, thereby increasing lungpower.

Breathing devices are also used for Inspiratory Muscle Training (IMT). Such IMT breathing devices generally have similar structure as PEP breathing devices, except the former restricts airflow during the inhale phase of a breathing cycle, while the latter restricts airflow during the exhale phase of a breathing cycle.

Therapeutic breathing devices, in general, may include motivational features—i.e., features that aid the user during therapy or provide feedback to the user regarding the progress or success of breathing exercises. Such motivational features include timers indicating the length of an exhale or inhale of breath, counters signifying a number of breaths a user takes, pressure gauges indicating the pressure exerted by the user's breath, balls or pistons that levitate with the user's breath to measure the strength or duration of a breath, or balloons that inflate and deflate as the user breathes. Such motivational features for use with breathing devices are discussed in U.S. Pat. Nos. 6,238,353 and 5,598,839 (actuatable indicators); U.S. Pat. Nos. 5,749,368 and 5,246,010 (pressure indicators); U.S. Pat. No. 5,865,172 (visual stimulator that moves and stops in response to breathing); and U.S. Pat. No. 6,463,928 (visual stimulator and musical feedback during breathing), incorporated herein by reference.

PEP breathing devices are generally categorized as either medical devices or physical fitness devices. Currently, some PEP breathing devices on the market are designed to facilitate breathing exercises or pulmonary therapy. Examples of such commercially available PEP breathing devices include: TheraPEP and Acapella (medical devices), DHD Healthcare Corporation, Canastota, N.Y. 13032; Power Lung fitness device, Power Lung Company, Houston, Tex.; and Sports Breather fitness device, alternately called The Breather, and marketed as a medical device from Sports Breather Company, Corpus Christi, Tex. More complex PEP breathing devices are also available for patients who are chronically ill and/or are hospitalized. The devices are usually provided for use in the hospital or are available by prescription by doctors.

Most prior art PEP breathing devices are fairly complicated and do not encourage the repetition required to adequately achieve maximum results for exercise and therapy. Moreover, none of the prior art PEP breathing devices offers a mouthpiece that encourages the user to utilize a pursed lip breathing technique, and therefore, the effectiveness of the therapy is limited. Further, motivational features used with prior art PEP breathing devices are also generally limited in scope and effectiveness.

A pursed lip breathing technique facilitates breathing exercises and makes the PEP therapy more effective. A pursed lip PEP breathing device has a specialized mouthpiece that causes the user to purse his or her lips. The act of pursing ones lips, i.e., drawing the lips together to become puckered as if one were going to whistle, prevents the cheeks from puffing out, thus ensuring that the therapeutic value of the exercise occurs in the bronchial airways and lungs instead of in the cheeks. Existing PEP breathing devices do not adequately encourage utilization of a pursed lip breathing technique.

### SUMMARY OF THE INVENTION

The present invention offers advantages and alternatives over the prior art by providing a pursed lip breathing device comprising a housing having an air channel defined therein and a mouthpiece attached to the housing for receiving a user's lips during breathing. The mouthpiece includes an aperture generally communicating with the air channel so that the user generates airflow in the air channel by breathing through the aperture. The mouthpiece also has an outer surface dimensioned so that the user's lips are pursed about the aperture during breathing. Means for restricting the airflow through the air channel are provided to induce a pressure on the user's lungs upon generation of the airflow by breathing.

The preferred breathing device may be provided with feedback means operatively connected to the air channel for providing responsive information, including entertainment and sensory feedback, to the user indicative of the user's breathing thereby encouraging the user during therapy and permitting the user to monitor the breathing. Such indicator means generally provide the user with ample motivation to perform and continue breathing exercises and therapy. In various embodiments envisioned by the present invention, the feedback means may include a controller operatively connected to an indicator unit, wherein the indicator unit may comprise a timer, a counter, or a light that operates in connection with the controller in response to an airflow in the air channel. Alternatively, the controller and the indicator unit may provide other sensory signals to the user, such as a sound, smell or taste during the user's breathing.

Alternatively, the feedback means may include a vibratory member disposed in the air channel that generates a sound in response to air flowing through the air channel; a pressure gauge that indicates the amount of pressure corresponding to the airflow in the air channel; and/or an actuatable member selectively actuatable between an extended position and a retracted position in reaction to airflow through the air channel. The present invention may also be directed to an embodiment of a pursed lip breathing device including an air channel configured as a maze and through which an object can be blown by the user's breathing.

In an alternative embodiment of the present invention, a pursed lip breathing device includes a housing having an air channel defined therein. The air channel has a first end for receiving an airflow from a user and a second opposing end. A mouthpiece is attached to the housing for receiving the user's lips during breathing and includes a stem having an aperture defined therein. The aperture generally communicates with the first end of the air channel so that the user generates airflow in the air channel by breathing through the aperture. The stem has an outer surface dimensioned so that the user's lips are pursed about the stem during breathing. A restrictor plate having a plurality of orifices of varying dimension is mounted for selective movement within the housing so that the orifices may be selectively aligned with the air channel to regulate the airflow capacity therethrough to thereby induce a pressure on the user's lungs upon generation of the airflow by breathing.

In another alternative embodiment of the present invention, a pursed lip breathing device includes a housing having an air channel defined therein. The air channel has a first end for receiving an airflow from a user and a second opposing end. A mouthpiece is attached to the housing for receiving the user's lips during breathing and includes a stem having an aperture defined therein. The aperture generally communicates with the first end of the air channel so that the user generates an airflow in the air channel by breathing through the aperture. The stem has an outer surface dimensioned so that the user's lips are pursed about the stem during breathing. An airflow adjustment member communicates with an opening in the air channel intermediate the first and second housing ends, and flexibly actuatable between a flexed position and a relaxed position so as to restrict the airflow capacity through the air channel. A porous limiting member is provided for defining the flexed position of the airflow adjustment member.

The present invention has the advantage of being used for PEP therapy and/or IMT therapy.

### BRIEF DESCRIPTION OF THE DRAWINGS

FIG. 1 is an isometric front view of an exemplary embodiment of a pursed lip breathing device in accordance with the present invention.

FIG. 2 is an isometric rear view of the pursed lip breathing device of FIG. 1.

FIG. 3 is a side view of the breathing tube assembly and the controller of the pursed lip breathing device of FIG. 1, with the housing removed.

FIG. 4 is a front view of the pursed lip breathing device of FIG. 1, i.e., view A of FIG. 1.

FIG. 5 is a cross sectional view of the pursed lip breathing device of FIG. 1 in an inhale position taken along the line BB of FIG. 4.

FIG. 6 is a cross sectional view of the pursed lip breathing device of FIG. 1 in an exhale position taken along the line BB of FIG. 4.

FIG. 7 is an isometric view of an alternative exemplary embodiment of a pursed lip breathing device illustrating a possible motivational feature which may be appropriate for children in accordance with the present invention.

FIGS. 8A and 8B are isometric views of alternative exemplary embodiments of pursed lip breathing devices in accordance with the present invention.

FIGS. 9A, 9B, 9C, 9D and 9E are isometric views of exemplary embodiments of mouthpieces for a pursed lip breathing device in accordance with the present invention.

FIG. 10 is a schematic illustrating the operation of a controller and an indicator unit to provide motivational features controlled to be responsive to an airflow during use of a pursed lip breathing device of the present invention.

### DETAILED DESCRIPTION OF THE PREFERRED EMBODIMENTS

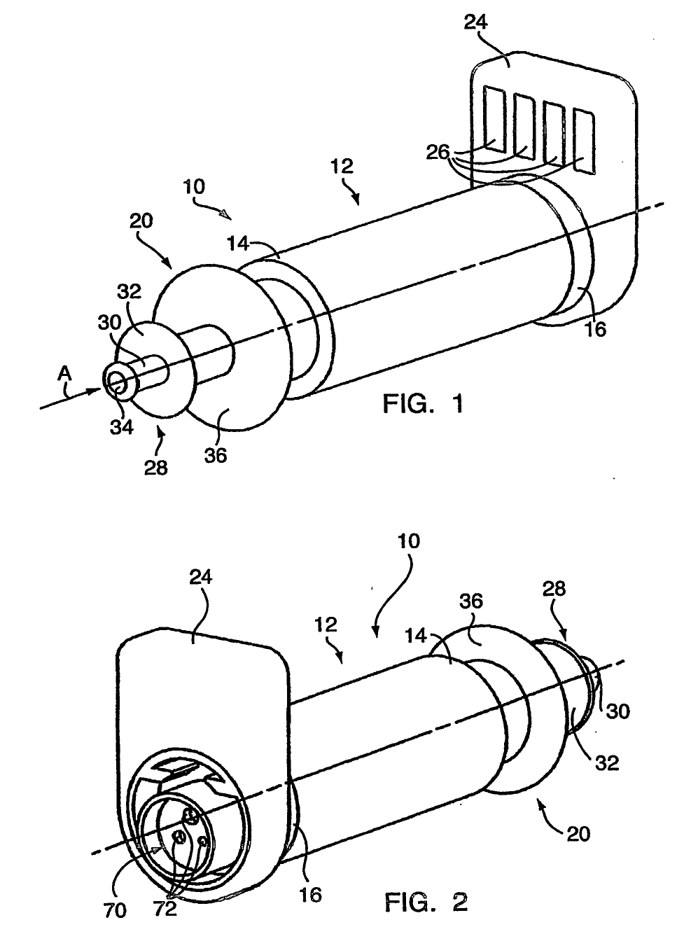

Referring to FIGS. 1-6, an exemplary embodiment of a pursed lip breathing device, is shown generally at 10. Preferably, the breathing device 10 is a pursed lip Positive Expiratory Pressure (PEP) breathing device that provides therapy to the user during an exhale phase of a breathing cycle, though the present invention may also be used as a pursed lip Inspiratory Muscle Training (IMT) breathing device to provide therapy during an inhale phase of a breathing cycle. In either case, the breathing device 10 controls the airflow generated by the user's breathing to induce pressure in the user's pulmonary system.

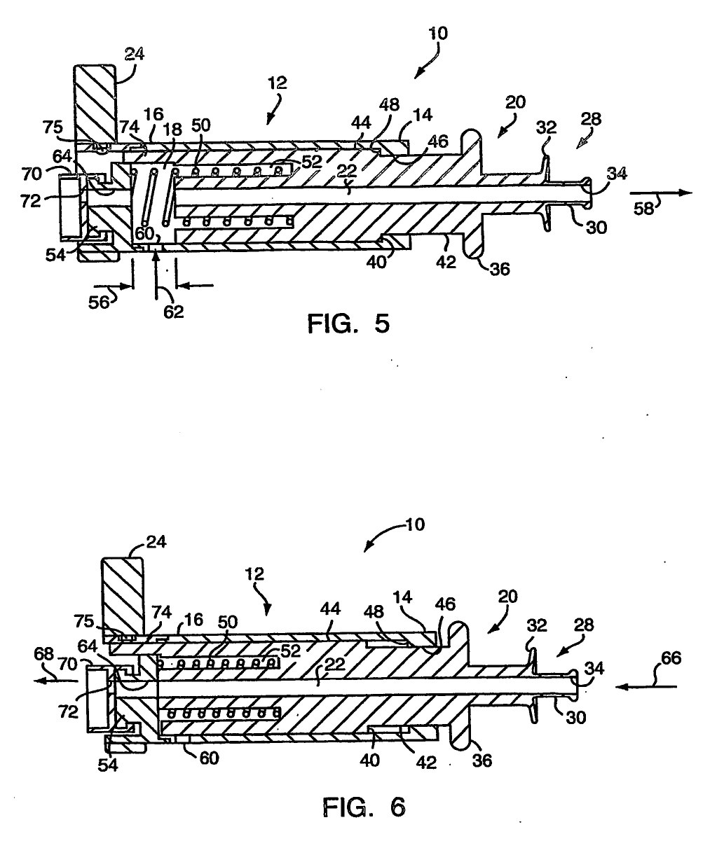

As illustrated, the pursed lip breathing device 10 includes a generally cylindrically shaped housing 12 having a first end 14 and a second end 16 and defining an interior cavity 18 preferably extending from the first end 14 to the second end 16. As shown in FIGS. 5 and 6, a breathing tube assembly 20 is slidably disposed in the housing cavity 18 through the first housing end 14, and defines an interior air channel 22 through which air flows as the user breathes into the device 10. The second housing end 16 is adapted to restrict the movement of the breathing tube assembly 20, as described in more detail below. Second housing end 16 is open or vented so that the airflow passes through the housing 12. Though the illustrated embodiment utilizes a cylindrical barrel as the housing 12, one skilled in the art will recognize that other shaped housings may also be used, e.g., a box-shaped housing having open opposing ends. Additionally, though the illustrated embodiment utilizes breathing tube assembly 20 to define an interior air channel 22, the air channel can be formed by other structure within the housing 12. For example, the housing cavity 18 can define an interior air channel through which air flows as the user breathes into the device 10. Further, the device 10 may include multiple air channels for exercising and regulating the user's breathing.

A controller 24 may also be fixedly mounted to the housing 12 to provide responsive information, motivational features, and/or sensory feedback to the user during breathing exercises. Depending on the desired information to be presented to the user, the controller 24 may operatively communicate with an indicator unit 26 for presentation of the desired information. As shown in FIGS. 1-6, the controller 24 is mounted to the housing 12 at the second housing end 16. In general, the controller can be adapted to be responsive to the airflow in the interior air channel 22, for example, by sensors signaling information about the airflow, or by a mechanical switching system that starts and stops the controller's operation. Operation of the controller 24 can then be coordinated with the indicator unit 26 to present responsive information and sensory feedback to the user. Operation of the controller 24 and the indicator unit 26 is described in more detail below.

A first end of the breathing tube assembly 20 includes a mouthpiece 28 integrally connected to the breathing tube assembly 20. Alternatively, the mouthpiece 28 may be removable and replaceable from the device 10, if, for example, the device 10 is to be used with multiple patients. The mouthpiece 28 illustrated in FIG. 1 includes a centrally disposed stem portion 30 extending outwardly from a mouth support 32. An aperture 34 is provided in the stem 30 and generally communicates with the air channel 22 so that the user generates an airflow in the air channel 22 by breathing through the aperture 34. The outer surface of the stem 30 is dimensioned and designed so as to encourage a user to purse his or her lips as he or she clamps his or her mouth around the stem 30 in order to make an air-tight seal while breathing into the mouthpiece 28. Typically, a properly sized mouthpiece 28 is substantially no greater than ¾ inch wide at the point where a user's lips engage the mouthpiece. This narrow outer dimension causes the user to taper and narrow the width of the user's mouth, and tighten his or her lips around the aperture 34 to create an air-tight seal. This pursing of the user's lips causes restriction in and of itself in the form of an action similar to that of whistling, which prevents puffing up the checks and induces an appropriate pressure in the user's pulmonary system.

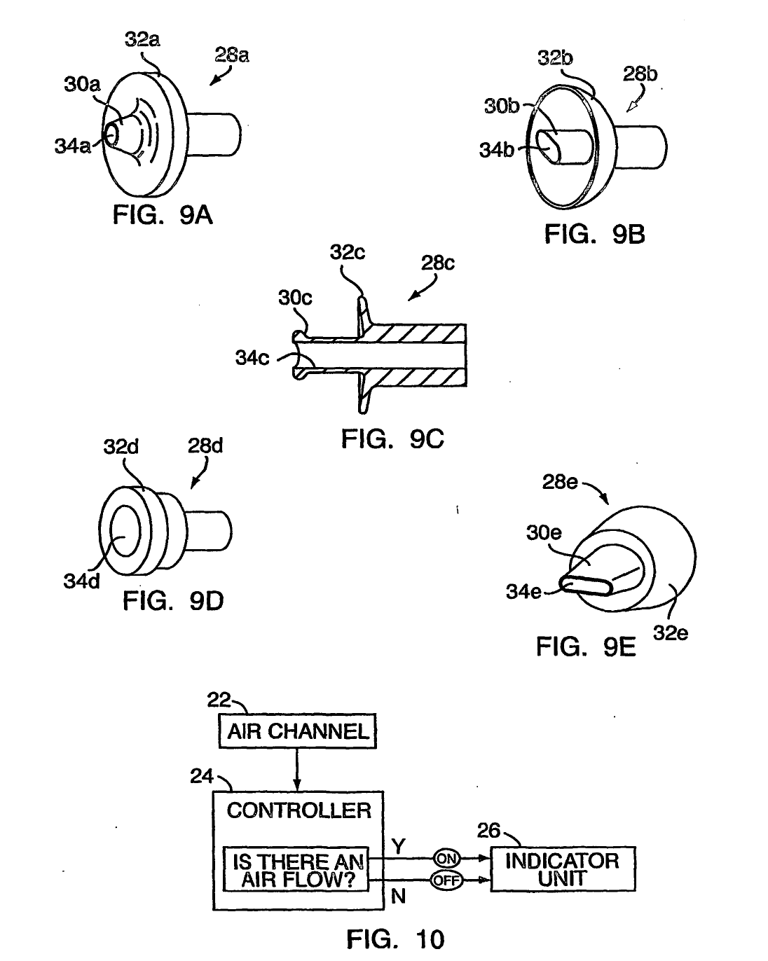

As shown in FIG. 1, the stem 30 has a substantially circular cross-sectional outside surface that is sized significantly smaller than a typical user's mouth. To breathe into the device 10, the user purses his or her lips about the stem 30 and against the mouth support 32. As long as the user maintains pursed lips during breathing, the therapeutic value of the device 10 will be enhanced. Though the stem 30 is shown in FIG. 1 as having a generally circular cross-section, one will recognize that other shapes may also be used—e.g., oval-shaped, cone-shaped, or tapered—provided the overall outer dimensions of the stem 30 are sufficiently small relative to a user's mouth to encourage the user to purse his or her lips during breathing into the device 10. A stem portion may not even be necessary, provided the mouthpiece 32 has a shape and dimensions that encourage the user to purse his or her lips about the aperture 34 during breathing. Alternate mouthpiece designs are illustrated in FIGS. 9A-9E, and described below.

The mouth support 32 is provided to further assist the user in pursing his or her lips during breathing. As illustrated in FIG. 1, the mouth support 32 has a generally concave dish shape, which is sized to receive, and preferably complement, a user's pursed lips in order to encourage a pursed lip breathing technique. That is, the lips of a user must be pursed to fit into the concave region of the mouth support 32 and around the stem 30. The mouth support 32 also provides a positive stop for the placement of a user's lips during operation and prevents the stem 30 from entering too far into the user's mouth. In mouthpiece embodiments having no stem portion, the shape and dimensions of the mouth support acts to encourage the user to purse his or her lips during breathing, as shown, for example, in FIG. 9D.

When operating the pursed lip breathing device 10, the user exhales through the aperture 34 in the mouthpiece 28. The mouthpiece 28 is connected to housing 12 so that the aperture 34 is generally aligned with the air channel 22 of the breathing tube assembly 20. As the user breathes through the aperture 34, an airflow is generated and permitted to pass through the air channel 22. The aperture 34 is sufficiently dimensioned to allow the user to inhale and exhale freely.

The mouthpiece 28 is made of bio-compatible material. The material is rigid enough so that it can be easily removed for washing and/or replacing, but soft enough to be comfortable and safe for a user's teeth and mouth. Additionally, the mouthpiece 28 may be constructed, in part or in whole, from a flavored, scented or edible material (e.g., candy).

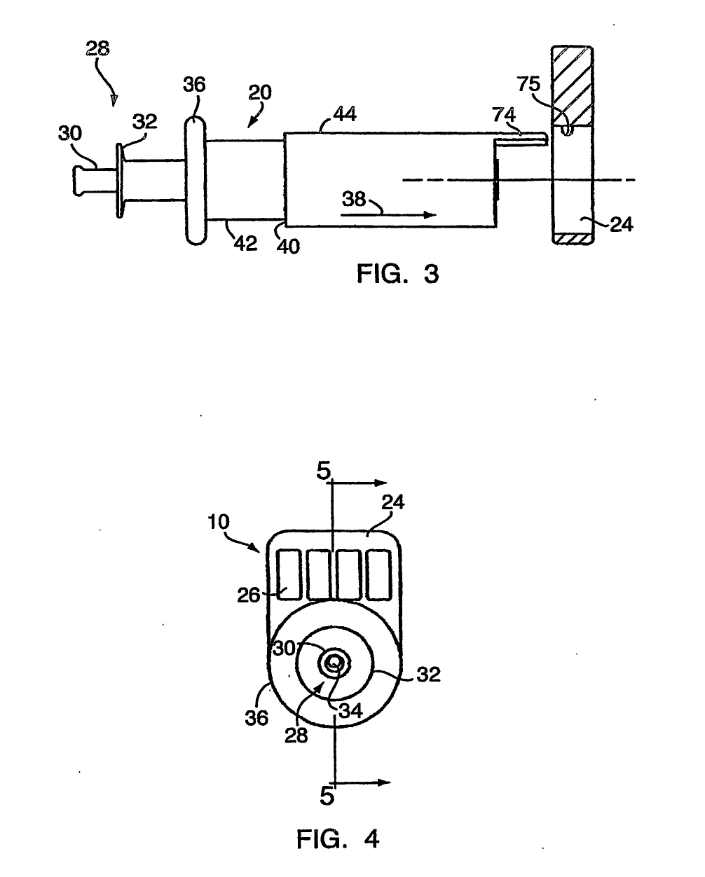

The breathing tube assembly 20 shown in FIGS. 1-6 also includes a thumb-pad 36. As shown, the thumb pad 36 is a disk that a user may press to slide the breathing tube assembly 20 within the housing 12, for example, in the direction of arrow 38 in FIG. 3. Referring to FIGS. 4-6, internal air channel 22 extends in the breathing tube assembly 20 rearwardly from the thumb pad 36. The outer surface of the breathing tube assembly 20 rearward of the thumb pad 36 is stepped so as to form a shoulder 40 dividing a minor outer diameter section 42 from an major outer diameter section 44 on the breathing tube assembly 20. The minor diameter section 42 is sized to slidably fit into an inside lip 46 of the first housing end 14 forming an inside housing shoulder 48. The major diameter section 44 is slidably engaged with the inside surface of the housing 12.

During operation, the breathing device 10 has two extreme positions, an inhale position (best seen in FIG. 5), and an exhale position (best seen in FIG. 6). A spring 50, or other resilient device or material, fits into a spring cavity 52 within the breathing tube assembly 20 and presses against an extension plate 54 mounted in the housing 12. The spring 50 biases the shoulder 40 on the breathing tube assembly 20 against the housing shoulder 48 and maintains a gap 56 between the extension plate 54 and the breathing tube assembly 20 when the breathing device 10 is in the inhale position. The gap 56 allows the airflow to freely bypass the air channel extension plate 54. Thus, the air will then easily flow through the air channel 22 while a user is inhaling (as indicated by the inhale airflow arrow 58). Optionally, one or more air holes 60 may be disposed in the housing 12 to further enhance inhaled airflow when the breathing device 10 is in the inhale position, as indicated by arrow 62. Air holes 60 may alternatively be configured as an inlet port for connection to a nebulizer, which can introduce medication to a patient while using the device 10.

In the exhale position, as shown in FIG. 6, the user presses on the thumb pad 36 to axially compress the spring 50 and slide the breathing tube assembly 20 towards the second housing end 16 to dose the gap 56 between the air channel extension plate 54 and the breathing tube assembly 20. Upon doing so, the air channel 22 of the breathing tube assembly 20 aligns and connects with an extension air channel 64 that extends though the center of the air channel extension plate 54 and out openings in the second housing end 16. As the user exhales through the device 10, an airflow passes through the air channel 22 and the extension air channel 64 (as indicated by the exhale airflow arrow 66) and exits out the second housing end 16 (as indicated by the exit airflow arrow 68). When the breathing tube assembly 20 is in the exhale position, the optional inhale air holes 60 are blocked by the major diameter section 44 of the breathing tube assembly 20.

For therapeutic operation of the breathing device 10, a pressure is induced on the user's lungs during breathing. For example, with a PEP device, the flow rate of expiratory air passing through the device 10 is controlled to induce pressure in the user's pulmonary system. More particularly, the flow rate is controlled by restricting the airflow capacity of the air channel in order to generate a back-pressure in the user's lungs. Referring to FIG. 2, a restrictor plate 70 is set into the second housing end 16 of the breathing device 10, and includes a plurality of orifices 72 of varying sizes, each of which may be selectively used to adjust and regulate the airflow capacity of the air channel 22. The restrictor plate 70 may be rotatable with respect to the housing so that any one of the three orifices 72 may be selectively aligned with the extension air channel 64 to regulate the airflow through the breathing tube assembly 20. The combination of predetermined inner diameters and lengths of the air channel 22 plus the selected orifice 72 on the restrictor plate 70 cause a desirable predetermined back pressure in the lungs of the user while exhaling. Although a pressure range of no greater than 20 cm H₂O is used in an exemplary embodiment of a PEP breathing device in accordance with the present invention, one skilled in the art will recognize that other desirable pressure ranges may also be used.

The restrictor plate 70 may be mounted on the housing 12 to abut the rear of the air channel extension plate 54, such that one of its variously sized orifices 72 aligns with the extension air channel 64 to provide additional back pressure regulation to a user's lungs. Alternatively, the restrictor plate 70 may be mounted at any axial location along the housing and actuatable to adjust which orifice 72 is aligned with the air channel 22 for airflow regulation.

Referring to FIGS. 3, 5 and 6, an electrically conductive contact tab 74 extends rearwardly from the distal, non-mouthpiece end of the breathing tube assembly 20. When in the inhale position, the electrical contact tab 74 does not make electrical contact with the controller 24. However, when in the exhale position, the electrical contact tab 74 electrically engages with the controller 24 to activate various types of informational and motivational features, and, if desired, present responsive information and feedback on the indicator 26. For example, the electrical contact tab 74 can engage a complementary electrical contact 75 operatively connected to the controller 24.

During breathing exercises and therapy, it is desirable to motivate and encourage the user. It is also useful to provide the user with feedback and other responsive information so that the user can assess the progress of the breathing exercises and therapy. For example, the user can be provided with information on the duration of a breath, the number of breaths taken during an exercise, the pressure generated by a breath, or the strength of a breath. Alternatively, the user can be entertained during breathing exercises or therapy. For example, the user's breathing can create a musical tone, activate a display, elicit a taste or smell, or require the performance of or completion of a task. This is especially helpful where the user is a child.

The above-identified motivational features can be accomplished using mechanical or electrical components. As noted, controller 24 may be mounted to the housing, as illustrated in FIGS. 1-6. The operation of the illustrated controller 24 is coordinated with the movement of the breathing tube assembly 20 between the inhale position (FIG. 5) and the exhale position (FIG. 6). When the user is exhaling through the device 10, the user presses on the thumb pad 36 and slides the breathing tube assembly 20 so that the air channel 22 aligns with and connects to the extension air channel 64. At the same time, the electrical contact tab 74 engages with the controller 24. This electrical connection instructs the controller 24 to commence a motivational event. Typically, the controller is operatively connected to the indicator unit 26. Such electronic structure and operation is schematically illustrated in FIG. 10. When the breathing tube assembly 20 is returned to the inhale position, for example by removing pressure from the thumb pad 36 and permitting the spring 50 to bias the breathing tube assembly 20 toward the first housing end 14, the electrical contact tab 74 is electrically disengaged from the controller 24. Alternatively, the controller 24 can be operable in response to signals from sensors positioned in the air channel to provide the controller with information regarding the airflow (e.g., presence of airflow; absence of airflow; exhale or inhale airflow).

The controller 24 and the indicator 26 are provided for motivating the user or for providing information and sensory feedback regarding the breathing exercises or therapy. The controller 24 is preferably adapted to commence an event in response the generation of an airflow in the breathing tube assembly 20. The controller 24 is further adapted to stop the event in response to the absence of airflow in the air channel 22. Alternatively, the controller 24 could be coordinated to provide different information depending on whether the user is exhaling, inhaling, or not breathing through the device 10. Further, the controller 24 can operate to reset at predetermined times or in response to certain circumstances associated with the user's therapy.

As illustrated in FIG. 10, the controller 24 operatively communicates with the indicator unit 26. Desired information regarding the exercise or therapy is provided on the indicator unit 26 as the user is breathing into the device 10. For example, the indicator unit 26 may be a timer that provides information on the duration of the user's breathing cycle, or counts-down the time remaining in a therapy session. The indicator unit 26 may also be a counter that counts, for example, a number of breaths taken during a therapy session. The information provided by the indicator unit 26 may also provide sensory feedback, including lights, sounds, tastes or smells. The controller 24 may contain also the electronic workings for utilizing light, sound and/or cartoon characters to be displayed on or by the indicator unit 26 for motivating the user. The controller 24 may also be designed to coordinate a flavored, scented or edible material provided in the mouthpiece 28 during breathing exercises or therapy.

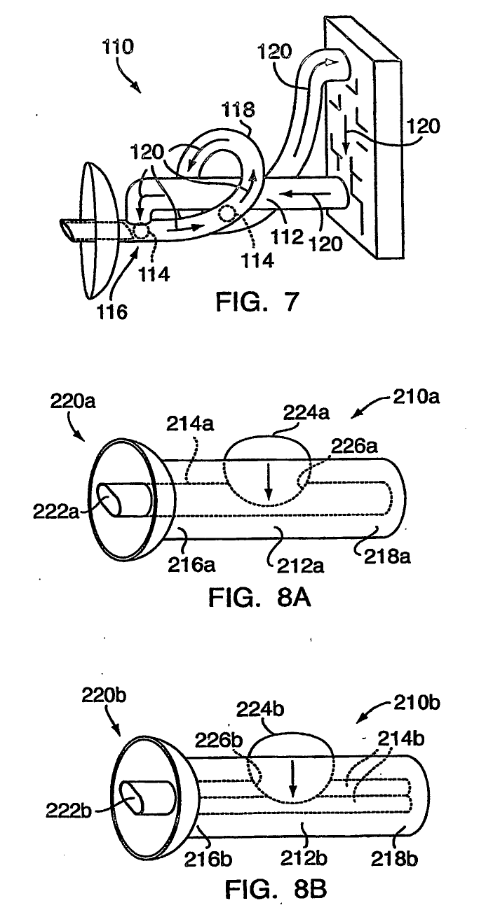

Though some of the above examples of motivational features are shown as utilized with the controller 24 and the indicator unit 26, one skilled in the art would recognize that many motivational features may be purely mechanical, or operable based upon and in response to the presence of an airflow in the air channel. For example, FIG. 7 illustrates an alternative embodiment of a pursed lip breathing device 110 in which an internal air channel 112 has a configuration of a maze in order to provide a motivational feature to the user during breathing. A ball 114, or other object, is provided in the air channel 112. The ball 114 can be moved or actuated under the influence of the user's breath from a start position 116, through a looped tubular track 118 of the maze and back to the start position 116 again. In FIG. 7, the path of the ball 114 is illustrated with directional arrows 120. The maze provides entertainment to motivate the user during breathing exercises. Additionally, the maze may be used to provide feedback information to the user regarding the progress of the breathing exercises. For example, the cycle time of the ball 114 through the air channel 112 can be timed to regulate the desired exhale/inhale rate of a user. During therapy, the user can try to see how many times he or she can advance the ball 114 through the maze during an exhale phase of the breathing cycle or during a therapeutic session. Data can be compared to previous attempts, or previous therapy sessions, to gauge the user's progress. In this particular exemplary embodiment, the only thing restricting the exhale of a user maybe be the dimensions of the air channel 112, though the breathing device 110 may be provided with additional components for restricting the airflow through the maze in accordance with a desired therapy session or technique, as described with respect to other embodiments herein.

Examples of other motivational or informational features that may be provided by the breathing device of the present invention include the use of a rubberized, elastic or flimsy material that, in a children's version of a pursed lip breathing device, may actuate in party-favor style from the pressure of user's exhale. Similarly, the device could include a balloon that inflates or deflates in conjunction with the user's breathing, such as that shown in U.S. Pat. No. 6,463,928, incorporated herein by reference. A breathing device may include a ball or piston in an air tube that floats or actuates under the influence of the user's breath during an exhale phase of a breathing cycle and returns to a rest position when no airflow is present, such as shown in U.S. Pat. No. 5,598,839, incorporated herein by reference. The user can try to maintain the ball in a section of the tube for as long as possible, or alternatively, the air tube may include indicia to indicate the strength of the user's exhale, such as shown in U.S. Pat. No. 5,246,010, incorporated herein by reference. The device may include a pressure gauge in fluid communication with the air channel in the breathing tube assembly to identify the amount of pressure corresponding to the airflow in the breathing tube assembly, such as shown in U.S. Pat. No. 5,749,368, incorporated herein by reference. The device, such as a reed or a whistle pea, may also include a vibratory member disposed in the air channel of the breathing tube assembly that generates a sound in response to air flowing through the air channel, such as shown in U.S. Pat. No. 6,463,928, incorporated herein by reference.

Various techniques, e.g., electrical, mechanical, written or otherwise, may also be utilized to motivate a user to vocalize (e.g., hum) while using a pursed lip breathing device of the present invention. Such vocalizations, while breathing through the device produce a vibratory quality to the throat that may have therapeutic value in some cases.

Alternative embodiments of a pursed lip breathing device, generally as reference numeral 210, are illustrated in FIGS. 8A and 8B. Like components of FIGS. 8A and 8B share reference numerals and are distinguished by alphabetical indicators suffixed thereto.

Whereas the embodiment of the pursed lip breathing device 10 of FIG. 1 is held in the fist of the user, the alternate designed breathing device 210 is held and operated with the user's fingertips. In FIG. 8A, the device 210a comprises a housing 212a defining an air channel 214a extending from a first housing end 216a to a second housing end 218a. A mouthpiece 220a, similar in design to that disclosed with respect to FIG. 1, is attached to the first housing end 216a and includes an aperture 222a generally communicating with the air channel so that an airflow is generated in the air channel 214a when the user breathes into the aperture 222a. Restriction of the airflow to generate a back-pressure in the user's lungs is caused by pressing the thumb (held on the bottom) and the forefinger/middle finger (held on the top) together. The airflow capacity of the air channel 214a is adjusted by depressing an airflow adjustment member 224a, made of a spongy, elastic, spring-like material, into a porous limiting member, illustrated as basket 226a, that restricts, but does not shut off airflow in the air channel 214a. The airflow adjustment member 224a retracts after use to its original, relaxed position to allow free airflow.

The basket 226a is porous to enable air to flow freely, not only through the air channel 214a, but also through the basket 226a itself when the airflow adjustment member 224a is in a retracted or non-depressed position. Basket 226a also provides a positive stop for the airflow adjustment member 224a when it is in the depressed position, preventing the airflow adjustment member 224a from overly restricting the airflow through air channel 214a.

Referring to FIG. 8B, an alternative multi-channeled embodiment of the pursed lip breathing device of FIG. 8A is shown generally at 210b. The alternative design is comprised of mouthpiece 220b, two or more air channels 214b, an airflow adjustment member 224b, and a basket 226b. The basket 226b may be porous or non-porous. Depressing airflow adjustment member 224b into the basket 226b restricts part or all of one channel 214b, or may completely close off one or more channels 214b (if there are greater than two channels) depending on the desired airflow requirements of the device 210b.

The embodiments of FIGS. 8A and 8B may also include structure that provides motivational features and/or sensory feedback to the user during breathing, such as the controller 24 and the indicator unit 26 illustrated with respect to the embodiment of FIGS. 1-6. As previously discussed with respect to other embodiments, additional electrical contacts (not shown) may be utilized to activate an optional controller and/or motivational aspects (not shown), or mechanical motivational features may be included to provide entertainment and sensory feedback for the user.

For any of the embodiments of the present invention, the mouthpiece may take one of several designs. Some examples of such designs are shown in FIGS. 9A-9E. Referring more particularly to FIGS. 9A, 9B, 9C, 9D and 9E, five exemplary embodiments of a pursed lip mouthpiece are respectively shown. Like components to the mouthpiece 28 shown in FIG. 1 are designated by the same reference numeral, but are distinguished by an additional alphabetical identifier suffixed thereto.

FIGS. 9A-9C generally illustrate mouthpieces including contoured dishes have a complementary shape to a user's pursed lips. In FIG. 9A, a mouthpiece 28a includes a contoured dish 32a as a mouth support. A centrally disposed aperture 34a is provided in a generally cone-shaped stem portion 30a. In FIG. 9B, a mouthpiece 28b includes a contoured dish 32b an oval-shaped or straw-like stem portion 30b having a centrally disposed aperture 34b. FIG. 9C illustrates a mouthpiece 28c of the general type shown and described in FIGS. 1-6, wherein the mouthpiece includes a stem 30c having a circular cross-section and an aperture 34c extending through the stem 30c, and a mouth support 32c having a concave dish shape for receiving the user's pursed lips. These mouthpiece designs generally require the user to purse his or her lips during breathing. The contoured dish mouth supports may be made of a malleable material that conforms to a user's lips as they are pressed against the dish. Additionally, the mouthpieces could enable hands-free operation enabling the pursed lip breathing device to be coupled with gym fitness equipment for an enhanced exercise program that encourages athletes to breathe properly while exercising at the gym.

FIG. 9D illustrates a mouthpiece 28d shaped generally like that of a trumpet mouthpiece. The mouthpiece 28d presents a narrow mouth support or dish 32d having a centrally disposed aperture 34d. The mouth support 32d requires a user to purse his or her lips in order to fit them within the dish to breathe through it. Unlike the other mouthpiece designs, this mouthpiece has no stem.

FIG. 9E illustrates a mouthpiece 28e having a stem portion 30e tapered from an aperture 34e to a mouth support 32e. The tapered shape of the stem 30e provides a more compact profile, and can be easily capped, for example, when the breathing device is not in use.

In the above embodiments of mouthpieces, the structure of each mouthpiece requires a user's lips to either fit over a generally circular or oval cross-sectionally shaped stem, and/or fit into a narrow passage. In either case, the dimensions of each mouthpiece design are sufficiently small relative to a user's lips such that the user is encouraged to purse his lips in order to make an airtight seal with the mouthpiece. During breathing, the user maintains his or her lips in a pursed state about the aperture so as to improve the efficiency of the breathing exercises.

Use of the above-described invention advantageously provides increased lung capacity by splinting airways open and mechanically clearing obstructed airways. Ensuring pursed lips during breathing also provides increased lungpower by developing the pulmonary musculature due to the repetitive nature of the exercise.

The present invention has a variety of potential uses and applications, including as a device for the enhancement of life for health-conscious individuals (yoga), and as a medical device for patients with chronic asthma, COPD, cystic fibrosis (CF), obstructive sleep apnea (OSA), and/or other pulmonary disorders. The present invention may also be used as a portable sports training device for athletes, or as a device to be utilized with portable or stationary gym equipment so that correct and therapeutic breathing becomes a part of normal exercise in the gym. When used as a medical device, the device can optionally have an opening in the bottom of the housing to accommodate a nebulizer that dispenses medication.

Though the above-described embodiments generally show a PEP breathing device, the present invention has equal applicability as an IMT breathing device. Such IMT breathing devices are generally the same as PEP breathing devices, except the former restricts airflow during the inhale phase of a breathing cycle, while the latter restricts airflow during the exhale phase of a breathing cycle.

While preferred embodiments have been shown and described, various modifications and substitutions may be made thereto without departing from the spirit and scope of the invention. Accordingly, it is to be understood that the present invention has been described by way of illustration and not limitation.

What is claimed is:

1. A pursed lip breathing device comprising:
a housing having an air channel defined therein;
a mouthpiece attached to the housing for receiving a user's lips during breathing,
wherein the mouthpiece includes an aperture generally communicating with the air channel so that the user generates an airflow in the air channel by breathing through the aperture, and a mouthpiece outer surface dimensioned so that the user's lips are pursed about the aperture during breathing; and
means for restricting the airflow through the air channel to thereby induce a pressure on the user's lungs upon generation of the airflow by breathing.

2. The pursed lip breathing device of claim 1, further comprising a breathing tube slidably disposed within the housing and defining the air channel;
wherein the breathing tube is selectively movable between a first position and a second position.

3. The pursed lip breathing device of claim 2, wherein the air channel operatively communicates with the restricting means when the breathing tube is in the second position such that air expelled from the user through the mouthpiece aperture will flow restrictively through the air channel to induce a pressure on the user's lungs during breathing; and
further wherein air expelled from the user through the mouthpiece aperture will flow unrestricted through the air channel and out the housing when the breathing tube is in the first position.

4. The pursed lip breathing device of claim 3, comprising:
an air channel extension plate mounted on the housing at an end distal from the mouthpiece, the air channel extension plate having an air channel extension extending therethrough and operatively communicating with the restricting means; and
a resilient member disposed between the breathing tube and the air channel extension plate to bias the breathing tube into the first position;
wherein, when the breathing tube is in the first position, the air channel and the air channel extension are separated by a gap that allows air expelled from the user to flow unrestricted through the air channel and out the housing, and, when the breathing tube is in the second position, the air channel aligns with the air channel extension so that air expelled from the user will flow through the air channel and the air channel extension and be restricted by the restricting means.

5. The pursed lip breathing device of claim 4, wherein the means for restricting the airflow through the air channel comprises a restrictor plate having a plurality of orifices of varying dimension, wherein the restrictor plate is mounted in the housing so that the orifices may be selectively aligned with the air channel to regulate the airflow capacity therethrough.

6. The pursed lip breathing device of claim 2, further comprising:
an electrical contact attached to the breathing tube; and
a controller attached to the housing and being operable to control an event representative of the user's breathing;
wherein the electrical contact is in electrical engagement with the controller and signals the controller to commence the event when the breathing tube is in the second position, and the electrical contact is in electrical disengagement from the controller and signals the control to stop the event when the breathing tube is in the first position.

7. The pursed lip breathing device of claim 1, further comprising feedback means operatively connected to the air channel for providing responsive information to the user indicative of the user's breathing thereby permitting the user to monitor the breathing.

8. The pursed lip breathing device of claim 7, wherein the feedback means comprises a controller attached to the housing and being operable to control an event representative of the user's breathing, and an indicator unit in operative communication with the controller for presentation of information regarding the user's breathing;
wherein the controller generates a first signal in response to an airflow through the air channel and generates a second signal in response to the absence of an airflow through the air channel, information being presented by the indicator unit upon receiving the first signal from the controller and stopping upon receiving the second signal from the controller.

9. The pursed lip breathing device of claim 8, further comprising an electrical contact associated with the air channel and selectively disposable, based on the user's breathing, between a first position when air is flowing in the air channel and a second position when no air is flowing in the air channel, such that the electrical contact electrically engages with the controller in the first position to start operation of the controller and the electrical contact electrically disengages from the controller in the second position to stop operation of the controller.

10. The pursed lip breathing device of claim 8, wherein the indicator unit is a light that turns on in response to an airflow in the air channel and turns of in response to the absence of an airflow in the air channel.

11. The pursed lip breathing device of claim 8, wherein the indicator unit is a timer that starts in response to an airflow in the air channel and stops in response to the absence of an airflow in the air channel.

12. The pursed lip breathing device of claim 8, wherein the indicator unit emits a noise in response to an airflow in the air channel.

13. The pursed lip breathing device of claim 7, wherein the feedback means comprises a vibratory member disposed in the air channel that generates a sound in response to air flowing through the air channel.

14. The pursed lip breathing device of claim 7, wherein the feedback means comprises a pressure gauge mounted to the housing that indicates the amount of pressure corresponding to the airflow in the air channel.

15. The pursed lip breathing device of claim 7, wherein the feedback means comprises an actuatable member communicating with the air channel and being selectively actuatable between an extended position and a retracted position in reaction to airflow through the air channel.

16. The pursed lip breathing device of claim 7, wherein the air channel has a maze configuration and the feedback means comprises an object that moves within the maze as the user breathes into the mouthpiece aperture.

17. The pursed lip breathing device of claim 16, wherein the maze configuration is a continuous loop so that the object can cycle through the maze during the user's breathing.

18. The pursed lip breathing device of claim 17, wherein the feedback means further comprises a counter for counting the number of times the object passes through the maze.

19. The pursed lip breathing device of claim 1, wherein the air channel has a first end generally communicating with the mouthpiece aperture, a second end, and an opening intermediate the first and second ends; and
wherein the means for restricting the airflow through the air channel comprises an airflow adjustment member attached to the housing and communicating with the opening to adjust the airflow through the air channel.

20. The pursed lip breathing device of claim 19, wherein the airflow adjustment member is selectively actuatable between a retracted position and an extended position wherein the airflow through the air channel is generally unrestricted when the adjustment member is in the retracted position and the airflow in the air channel is restricted to varying degrees as the adjustment member is actuated between the retracted position and the extended position.

21. The pursed lip breathing device of claim 19, wherein the airflow adjustment member comprises a restrictor plate having a plurality of orifices of varying dimension, wherein the restrictor plate is mounted in the housing so that the orifices may be selectively aligned with the air channel to regulate the airflow capacity therethrough.

22. A pursed lip breathing device comprising:
a housing having an air channel defined therein, the air channel having a first end for receiving an airflow from a user and a second opposing open end;
a mouthpiece attached to the housing for receiving the user's lips during breathing,
wherein the mouthpiece includes mouth support portion and a stem portion having an aperture defined therein, the aperture generally communicating with the first end of the air channel so that the user generates an airflow in the air channel by breathing through the aperture, and the stem portion having an outer surface dimensioned so that the user's lips are pursed about the stem portion during breathing; and
a restrictor plate having a plurality of orifices of varying dimension, wherein the restrictor plate is mounted for selective movement within the housing so that the orifices may be selectively aligned with the air channel to regulate the airflow capacity therethrough to thereby induce a pressure on the user's lungs upon generation of the airflow by breathing.

23. The pursed lip breathing device of claim 22, further comprising feedback means operatively connected to the air channel for providing responsive information to the user indicative of the user's breathing thereby permitting the user to monitor the breathing.

24. A pursed lip breathing device comprising:
a housing having an air channel defined therein, the air channel having a first end for receiving an airflow from a user and a second opposing open end;
a mouthpiece attached to the housing for receiving the user's lips during breathing,
wherein the mouthpiece includes mouth support portion and a stem portion having an aperture defined therein, the aperture generally communicating with the first end of the air channel so that the user generates an airflow in the air channel by breathing through the aperture, and the stem portion having an outer surface dimensioned so that the user's lips are pursed about the stem portion during breathing; and
an airflow adjustment member communicating with the opening to adjust the airflow through the air channel to thereby induce a pressure on the user's lungs upon generation of the airflow by breathing, the airflow adjustment member being flexibly actuatable between a flexed position and a relaxed position,
wherein the airflow through the air channel is unrestricted when the airflow adjustment member is in the relaxed position and the airflow in the air channel is restricted to varying degree as the airflow adjustment member is flexibly actuated between the relaxed position to the flexed position; and
the device further comprising a limiting member positioned relative to the opening for defining the flexed position of the airflow adjustment member.

25. The pursed lip breathing device of claim 24, further comprising feedback means operatively connected to the air channel for providing responsive information to the user indicative of the user's breathing thereby permitting the user to monitor the breathing.

26. A mouthpiece for use with a therapeutic breathing device that restricts airflow therethrough to induce a pressure on the user's lungs during breathing, the mouthpiece comprising:
a mouth support portion for receiving a user's lips during breathing; and
an aperture extending through the mouth support portion;
wherein the mouth support portion is dimensioned so that the user's lips are pursed about the aperture during breathing.

27. The mouthpiece of claim 26, further comprising a stem portion extending from the mouth support and defining the aperture.

28. The mouthpiece of claim 26, at least a portion of the mouthpiece being edible.

29. The mouthpiece of claim 26, at least a portion of the mouthpiece being flavored.
